(19) United States
(12) Patent Application Publication
(10) Pub. No.: US 2012/0056102 A1
(43) Pub. Date: Mar. 8, 2012

(54) Ultraviolet Light Sanitizing Method and Apparatus

(76) Inventors: Kenneth A. Stanley, Okeechobee, FL (US); Bruce C. Armstrong, Juipter, FL (US); Thomas F. Ruggirello, Fort Pierce, FL (US); Troy Daniel Smith, Stafford, TX (US); Kenneth A. Stanley, JR., Fort Saint Lucie, FL (US)

(21) Appl. No.: 13/225,253

(22) Filed: Sep. 2, 2011

Related U.S. Application Data
(63) Continuation-in-part of application No. 12/291,077, filed on Nov. 6, 2008, now abandoned.

(51) Int. Cl. G21K 5/08

(52) U.S. Cl. 250/455.11

(57) ABSTRACT

A unit, system, and method for disinfecting or sterilizing the entire surface area of an item. The system includes at least one ultraviolet light source producing ultraviolet light for disinfecting the item. In addition, the system including a cavity housing the ultraviolet light source, the cavity having a reflective interior for redirecting light produced by the at least one ultraviolet light source. Furthermore, the system including a shelf positioned above a bottom portion of the cavity to support the item, the shelf capable of passing light produced by the at least one ultraviolet light source there through to disinfect an entire surface area of the item.

### CROSS-REFERENCE TO RELATED APPLICATION

This patent application is a continuation-in-part application of U.S. patent application Ser. No. 12/291,077, filed Nov. 6, 2008 entitled Ultraviolet Light Sanitizing Method and Apparatus which application is incorporated in its entirety here by this reference.

### TECHNICAL FIELD

The present application relates to microbes, and more particularly, to a system for disinfecting the entire surface area of an object using ultraviolet light and a surface capable of passing the ultraviolet light therethrough.

### BACKGROUND ART

Each year, thousands of children and adults get sick as a result of contaminated food. Microbes gain access to food virtually at any stage of the food's manufacture, from harvest of the raw materials through post-harvest storage, processing, and distribution. Good practice through the food chain from raw material to finished product is intended to ensure that the food that reaches the consumer is wholesome and above all, safe to eat, yet outbreaks of illnesses attributable to food-borne pathogenic microbes still arise, implying microbial contamination at some link in the chain before the food reaches the consumer.

Cooking sterilizes food using heat to kill the microbes. In general applications of heat, water with the temperature of about 74° C. (165° F.) or higher sterilize the food. Nonetheless, food often becomes contaminated thereafter causing sickness and possibly, death to those who are more susceptible to food poising, such as the very young or old, or those whose immune system is already compromised.

Ordinary utensils, bottles, and everyday items coming in contact with those susceptible to illness may also retain microbes making them sick and possibly, even causing death. Heat and chemicals often provide the best option to sterilize these items. Nonetheless, using these results in releasing harmful environmental pollutants into the air.

### DISCLOSURE OF THE APPLICATION

This summary is provided to introduce a selection of concepts in a simplified form that is further described below in the DETAILED DESCRIPTION OF THE APPLICATION. This summary is not intended to identify key features of the claimed subject matter, nor is it intended to be used as an aid in determining the scope of the claimed subject matter.

In accordance with one aspect of the present application, a disinfection unit is presented. The disinfection unit includes at least one ultraviolet light source producing ultraviolet light for disinfecting an item. In addition, the disinfection unit includes a cavity housing the ultraviolet light source, the cavity having a reflective interior for redirecting the ultraviolet light produced by the at least one ultraviolet light source. Furthermore, the disinfection unit includes a shelf positioned above a bottom portion of the cavity to support the item, the shelf capable of passing light produced by the at least one ultraviolet light source therethrough to disinfect an entire surface area of the item.

In accordance with another aspect of the present application, a system for sterilizing an item is presented. The system includes a chamber and a light source located within the chamber. In addition, the system includes a member positioned within the chamber to allow ultraviolet light produced from the light source to disinfect an entire surface area of the item by allowing the ultraviolet light to pass through.

In accordance with yet another aspect of the present application, a method is presented. The method includes placing an item on a surface within a chamber having reflective walls. In addition, the method includes applying ultraviolet light from a single light source to the item, wherein the ultraviolet light directly strikes the item and further, indirectly strikes the item through the surface or redirected off the chamber walls allowing the ultraviolet light to strike the item at three hundred sixty degrees using the single light source.

### BRIEF DESCRIPTION OF THE DRAWING(S)

For a better understanding of the present application, reference is made to the below-referenced accompanying Drawing(s). Reference numbers refer to the same or equivalent parts of the present application throughout the several figures of the Drawing(s).

FIG. 1 is a diagram showing a side elevated view of an exemplary disinfection unit having a rectangular box configuration in accordance with one aspect of the present application;

FIG. 2 is an illustration showing a front view of the exemplary disinfection unit having the rectangular box configuration in accordance with one aspect of the present application;

FIG. 3 depicts exemplary placements of illustrative ultraviolet light sources within the rectangular box configuration in accordance with one aspect of the present application;

FIG. 4 shows typical paths taken by ultraviolet light produced from the ultraviolet light sources within the rectangular box configuration in accordance with one aspect of the present application;

FIG. 5 is an illustration showing a front view of an exemplary disinfection unit having a spherical configuration in accordance with one aspect of the present application;

FIG. 6 depicts exemplary placement of an illustrative ultraviolet light source within the spherical configuration in accordance with one aspect of the present application; and

FIG. 7 shows typical paths taken by ultraviolet light produced from the ultraviolet light sources within the spherical configuration in accordance with one aspect of the present application.

### DETAILED DESCRIPTION OF THE APPLICATION

The detailed description set forth below in connection with the appended drawings is intended as a description of presently-preferred embodiments of the application and is not intended to represent the only forms in which the present application may be constructed and/or utilized. The description sets forth the functions and the sequence of steps for constructing and operating the application in connection with the illustrated embodiments. It is to be understood, however, that the same or equivalent functions and sequences may be accomplished by different embodiments that are also intended to be encompassed within the spirit and scope of this application.

Generally described, the present application relates to an apparatus for microbial decontamination. More specifically, the present application relates to a disinfecting unit having a member allowing ultraviolet light to pass through, the ultraviolet light capable of striking an item placed on the member at three hundred sixty degrees around the item. As a result, the ultraviolet light disinfects or sterilizes the entire surface area of the item. In an illustrative embodiment, an item is placed on a surface within a chamber having reflective walls. A single ultraviolet light source, placed within the chamber, generates ultraviolet light capable of striking the entire surface area of the item either directly or indirectly. Light not only strikes the item directly, but can pass through the transparent glass surface and strike the item. Furthermore, the reflective walls of the chamber redirect any ultraviolet light into the item, thus disinfecting or sterilizing the entire surface area of the item. The disinfecting unit provides an attractive alternative to radiation or pasteurization.

Central to the disinfecting unit is the shelf, member, or surface providing support for the item. While shelf, member, or surface are described through out this application, one skilled in the relevant art will appreciate that such terms may be interchangeable together or with other terms representing the same concept.

While the term “item” is described generally throughout this application, item can refer to, but is not limited to, food, clothes, surgical tools, baby bottle tops, hairbrushes, etc. Depending on the size of the disinfecting unit itself, a variety of different types of items can be placed within the disinfecting unit. Furthermore, more than one item can typically be placed within the unit, the unit still capable of disinfecting or sterilizing the entire surface area of the item. Most items can be placed within the numerous embodiments and aspects described throughout this application.

In addition, the term cavity may be interchangeably used with the term chamber. Also, the terms disinfecting unit may also be referred to as system through the application. One skilled in the relevant art will appreciate that there are numerous terms that may be exchanged and as such, this application is not limited to those terms.

Ultraviolet light is useful for sterilizing and disinfecting surfaces. Ultraviolet light, having a wavelength sufficient to break down microbes, can be used in a variety of applications, such as food, air, and water purification. Effectively, the ultraviolet light destroys the nucleic acids in the microbes so that their DNA is disrupted by the ultraviolet light radiation, which is a form of ionizing radiation, removing their reproductive capabilities and killing them.

Dependent on the source of ultraviolet light, disinfecting or sterilizing times may vary. In one embodiment, items are exposed to ultraviolet radiation from about one to thirty seconds. In other embodiments, items are radiated with the ultraviolet light from about thirty seconds to the time needed to disinfect item(s). Exposing items to such conditions generates little or no increase in temperature of the item.

Ultraviolet light having wavelengths from about one hundred nanometers to about four hundred nanometers are typically used in this application. It has been found that these wavelengths effectively eliminate microbes on the entire surface area of the item. More preferably, however, ultraviolet light having wavelengths of about two hundred fifty three point seven nanometers have been used to eliminate microbes. One skilled in the relevant art can appreciate that many different types of light may be used to effectively eliminate microbes and the application, as presented, is not limited to the use of ultraviolet light.

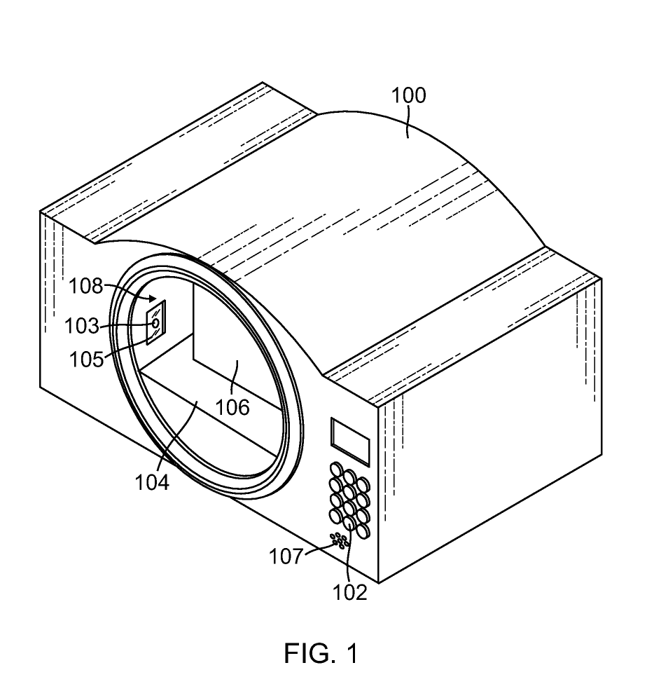

To effectively eliminate microbes on an item, multiple configurations of using ultraviolet light are presented. The embodiments presented in this application allow for the ultraviolet light source to strike the item around three hundred sixty degrees thereby completely removing microbes or other infectious germs from the entire surface area of the item. FIG. 1 is a diagram showing a side elevated view of an exemplary disinfection unit 100 having a rectangular box configuration in accordance with one aspect of the present application. Preferably, the exterior of the disinfection unit 100 has a means for mounting the unit 100 on a countertop, tabletop, or any other flat surface, such as a flat bottom, a flat top, or a plurality of feet projecting from the bottom. Disinfection unit 100 can have a bubble top for a more sleek style or any other shape thereof.

Computing system 102 provided on the face of disinfection unit 100 allows the user to view, select, and enter in a variety of options. Such options can include a clock, time of ultraviolet light exposure, clear function, enter function, and numerical digits for entering in the time for ultraviolet exposure. The computer system can take the form of an entirely hardware embodiment or an embodiment containing both hardware and software elements.

Continuing with FIG. 1, enclosed within disinfection unit 100 is cavity 108. Cavity 108 houses item 106. While a circular opening to cavity 108 is provided, the opening may come in a variety of different forms allowing item 106 to be inserted into the disinfection unit 100. This may include, but is not limited to, a square, rectangle, or any other type of shape. Provided on the opening to cavity 108 may be a rubber seal for preventing ultraviolet light from escaping cavity 108.

Disinfection unit 100 also contains shelf 104. To provide three hundred sixty degrees of exposure to item 106 from the ultraviolet light, shelf 104 may be made of glass, but can be made of a variety of other materials. Shelf 104 is generally made of materials capable of passing and not affecting ultraviolet light. Shelf 104 can generally be made of quartz, Teflon AF, or Teflon FEP. Furthermore, shelf 104 can be made of soft glass. Each of these materials allows ultraviolet light to pass therethrough without interfering or altering the ultraviolet light. While preferred embodiments have been disclosed, shelf 104 may incorporate other materials that allow ultraviolet light to pass through.

Although a single shelf 104 is provided for in FIG. 1, multiple shelves may be incorporated within cavity 108. These shelves 104 may be positioned above and/or below one another. Alternatively, walls made of the same material as shelf 104 along with the shelves 104 can be used to separate items 106 by forming cubicles within cavity 108. Separate items 106 can be stored in different cubicles as each item 106 may require different treatment times. As such, shelves 104 may be placed horizontally and walls may be placed vertically in any combination thereof. In some embodiments, the shelf may be adjustable within the cavity to accommodate items 106 of different sizes.

To expose item 106 from every angle, item 106 is placed on top of shelf 104 allowing the underside of item 106 to be exposed to ultraviolet light. Shelf 104, as shown in FIG. 1, is typically placed above a bottom portion of the cavity so as to form a subcavity directly beneath the shelf 104. In the preferred embodiment, shelf 104 can be supported by bracings on the side of cavity 108. This eliminates potential structures that can block or alter the transmission of the ultraviolet light. Alternatively, shelf 104 can be supported through other means including support from the bottom of cavity 108 or on top of cavity 108. The support may also be made from the same material as the shelf 104 so as to minimize obstructing the transmission of the ultraviolet light.

Shelf 104 can also be rotated within cavity 108 relative to substantially the entire housing (except the point of rotation) by being mounted on pins 109 at opposite ends of the shelf 104 along a midline m of the shelf 104. A motorized mechanism 111 controlled by the computing system 102 may be used to rotate the shelf 104 about the pins 109. Other mechanisms for rotating the shelves can be used. Through rotation, item 106 can be displaced relative to cavity 108, preferably substantially radially or substantially arcuately, so as to expose contact points on the item 106 to ultraviolet radiation. Rotation and displacement not only help to eliminate untreated areas, but also help to make the treatment more uniform across the entire surface area of item 106, helping to reduce the required treatment times.

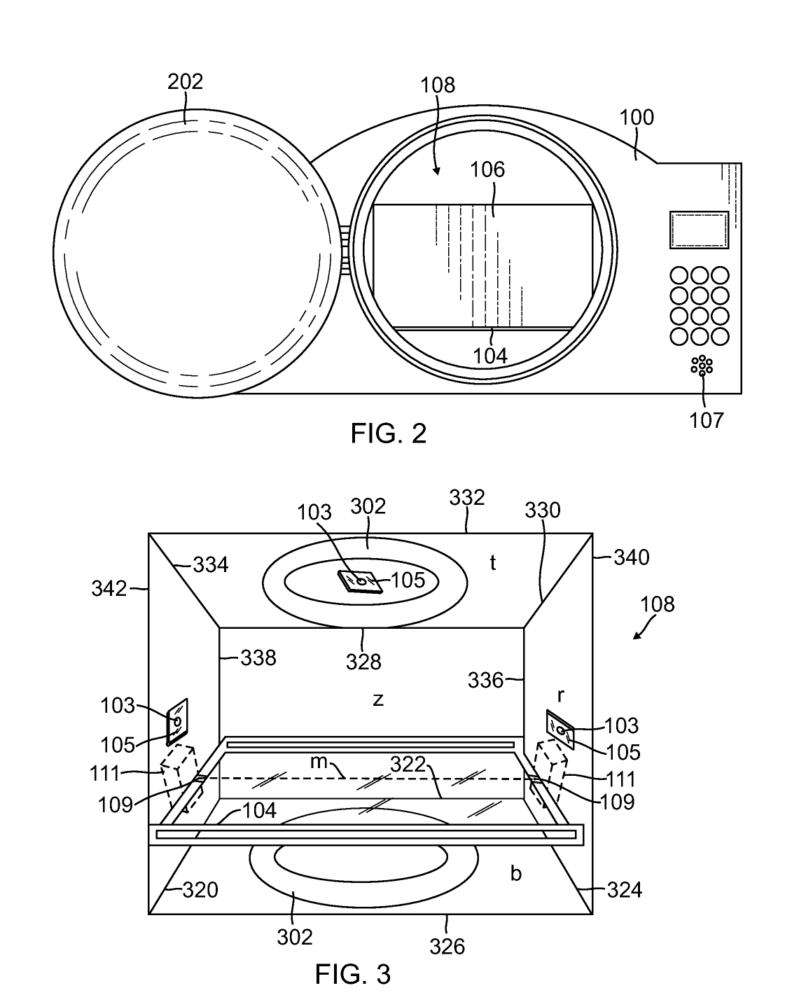

A front view of exemplary disinfection unit 100 having the rectangular box configuration is depicted in FIG. 2. However, the exterior of the disinfection unit 100 can be any other shape. In preferred embodiments, door 202 coupled to disinfection unit 100 can be used to close and open disinfection unit 100 preventing ultraviolet light from escaping or exiting cavity 108. In the rectangular box configuration, the interior surface of door 202 that is exposed to the cavity of disinfection unit 100 is typically flat. However, the interior of the door surface can be any other shape. In particular, the interior of the door surface may be curved or arcuate in shape to be consistent with an embodiment in which the interior walls of the housing are curved or arcuate so as to define a spherical cavity 108.

Typically, items 106 are placed within cavity 108 and door 202 is shut to prevent ultraviolet light from escaping disinfection unit 100. Alternatively, disinfection unit 100 may not include door 202. In this embodiment, items 106 can be transported in and out of the disinfection unit 100 using an automated process. This automated process can include a conveyer belt made of the same glass as shelf 104 or any other material that allows ultraviolet light to pass.

Preferred dimensions of disinfection unit 100 have a depth of fourteen inches, a height of eleven point ninety-nine inches, and a width of twenty-one point thirty-five inches. These dimensions are exemplary and do not limit the scope of the application. One skilled in the relevant art will appreciate that different types of dimensions may exist for disinfection unit 100.

In some embodiments, to create cavity 108, bottom wall b, top wall t, left side wall l, right side wall r, back wall z, and a front wall (not shown) connected to door 202 are used to form a rectangular box configuration. In some embodiments, the entire front wall may function as the door. Each wall may be planar and coupled to at least another wall. Bottom wall b has edges 320, 322, 324, and 326. Edge 320 is coupled to left side wall l. Edge 322 is coupled to back wall z. Edge 324 is coupled to right side wall r. Edge 326 is coupled to the front wall.

Top wall t has edges 328, 330, 332, and 334. Edge 328 is coupled to back wall z. Edge 330 is coupled to right side wall r. Edge 332 is coupled to the front wall. Edge 334 is coupled to left side wall l. Back wall z has edges 322, 328, 336, and 338. Edge 322 is coupled to bottom wall b. Edge 328 is coupled to top wall t. Edge 336 is coupled to right side wall r. Edge 338 is coupled to left side wall l. Right side wall r has edges 324, 330, 336, and 340. Edge 324 is coupled to bottom wall b. Edge 330 is coupled to top wall t. Edge 336 is coupled to back wall z. Edge 340 is coupled to the front wall.

Left side wall l has edges 320, 334, 338, and 342. Edge 320 is coupled to bottom wall b. Edge 334 is coupled to top wall t. Edge 338 is coupled to back wall z. Edge 342 is coupled to the front wall connected to door 202. The front wall has edges 326, 332, 340, and 342. Edge 326 is coupled to bottom wall b. Edge 332 is coupled to top wall t. Edge 340 is coupled to right side wall r. Edge 342 is coupled to left side wall l.

Continuing with FIG. 3, shelf 104 is placed above bottom wall b allowing ultraviolet light to strike items 106 that are placed on shelf 104. While shelf 104 extends from left side wall l to right side wall r, this is not limiting. As such, shelf 104 may be supported by bottom wall b, back wall z, or even top wall t.

Cavity 108 also houses at least one ultraviolet light source 302. Placement of ultraviolet light sources 302 within cavity 108 may vary and are not limited to the positions described herein. As depicted in FIG. 3, one ultraviolet light source 302 is coupled to top wall t, while another ultraviolet light source 302 is coupled to bottom wall b. One skilled in the relevant art will appreciate that the larger the number of ultraviolet light sources 302 used, the more energy required.

Each wall of cavity 108 can include reflective material to redirect ultraviolet light from ultraviolet light sources 302. The reflective material can be made of a thin polished layer of metal, such as aluminum, which is deposited on glass substrates. Metals used determine the reflective characteristics. Aluminum is typically the cheapest and most common reflective material. Other reflective materials may include, but are not limited to, silver and gold. Silver, which has a reflectivity of about ninety-five percent to about ninety-nine percent can reflect ultraviolet spectral regions. Gold, which has a reflectivity of about ninety-eight percent to about ninety-nine percent, can reflect light which has wavelengths below five hundred fifty nanometers. Increasing and decreasing the density and thickness of the metals used also determines the reflectivity of the walls. Preferably, the metals are grounded.

In other embodiments of the present application, non-reflective walls may be used within cavity 108. Typically, this embodiment would require a plurality of light sources in order for the entire surface area of item 106 to be exposed to ultraviolet light. Still yet, some of the walls will allow light to pass through, while others do not.

Instead of using reflective metals as described above, reflective coatings can be placed on the walls. In exemplary embodiments, dielectric coatings having a different refractive index to the substrate may be used. Dielectric coatings can include magnesium fluoride, calcium fluoride, and various metal oxides, which are deposited onto a substrate. By carefully choosing the exact composition, thickness, and number of layers, reflectivity and transmitivity of the coating can produce any desired characteristic.

Through the dielectric coatings, reflectivity can be increased to greater than ninety-nine point ninety-nine percent producing a high-reflector coating. The level of reflectivity can also be tuned to any particular value, for instance to produce a mirror that reflects ninety percent and transmits ten percent of the light that falls on it, over some range of wavelengths.

Multiple implementations of producing reflective walls for disinfecting unit 100 can be used and as such, is not limited to the discussion presented above. Furthermore, ultraviolet sources 302 may be shaped and formed in many different ways. Still yet, ultraviolet light sources 302 may be embedded into the walls or rise above the walls. Thus, the ultraviolet light sources 302 may come in a variety of different shapes, sizes, and placed in a variety of locations with cavity 108.

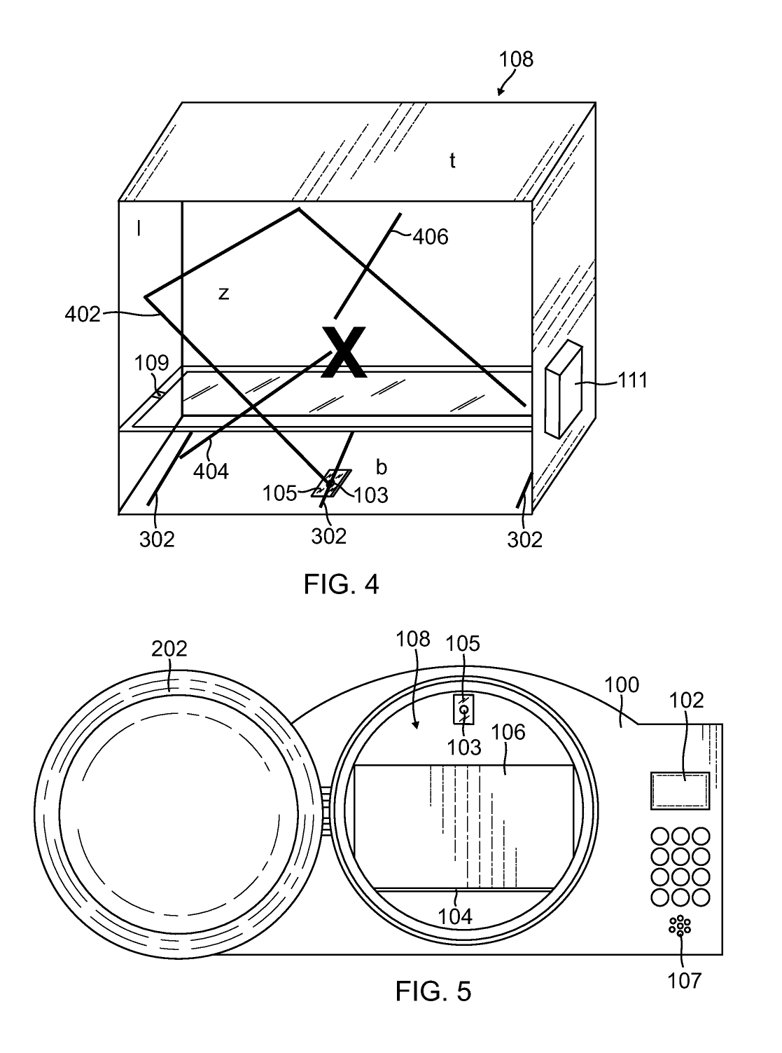

Examples of paths taken by ultraviolet light produced from the ultraviolet light sources 302 within the rectangular box configuration of cavity 108 are shown in accordance with one aspect of the present application depicted in FIG. 4. While the ultraviolet light is depicted in beams, ultraviolet light is understood to contain properties of both waves and particles. The beams can directly strike item 106. In addition, the beams can strike item 106 through the transparent glass surface 104 or be redirected off the cavity 108 walls allowing the ultraviolet light to eventually strike item 106. Through the combination of glass surface 104 and the walls, item 106 can be hit with ultraviolet light from all angles using a single ultraviolet light source 302. For purposes of illustration, three separate beams 402, 404, and 406 will be followed within cavity 108 of disinfecting unit 100.

In one illustrative beam, beam 402 begins at the middle ultraviolet light source 302 coupled to bottom wall b of cavity 108. Beam 402 is projected towards glass 104. When beam 402 reaches glass 104, beam 402 goes through glass 104 as if glass 104 was transparent. Because of the chemical properties of glass 104, beam 402 is only slightly or not affected in any manner.

Thereafter, beam 402 hits or strikes left side wall l. Left side wall l redirects beam 402 based on the incoming angle and reflects beam 402 towards top wall t. Because item 106 has not been reached, beam 402 is again redirected off top wall t towards right side wall r. Beam 402 continues to be redirected off the walls and through glass 104 until beam 402 finally strikes the surface of item 106.

Exemplary beam 404 begins at the left ultraviolet light source 302 coupled to bottom wall b of cavity 108. Beam 404 is projected towards glass 104. When beam 402 reaches glass 104, beam 402 goes through glass 104 striking a bottom portion of item 106. Through similar directed beams, the bottom portion of the entire item 106 can receive ultraviolet light from ultraviolet light source 302.

Illustrative beam 406 begins from ultraviolet light source 302 coupled to top wall t of cavity 108. Beam 406 is projected towards item 106. Thereafter, beam 406 directly strikes item 106. Beams can also be redirected or reflected off back wall z and the wall on door 202. Through exemplary beam 402, beam 404, and beam 406 light can strike item 106 from all angles, thereby disinfecting or sterilizing the entire surface area of item 106.

In a preferred embodiment, a single ultraviolet light source 302 is used taking advantage of the reflective walls and glass shelf 104. This greatly lowers the cost as it reduces the hardwiring needed to operate multiple ultraviolet light sources 302. Furthermore, the costs can be realized when only one ultraviolet light source 302 needs to be replaced.

Through this configuration, items 106 may also be scattered along glass shelf 104 and do not have to be directly placed within the middle. The combination of glass shelf 104 and the reflective side walls allow for items to be struck around three hundred sixty degrees even though items 106 are not placed within the middle of the housing.

In alternative embodiments, a front view of an exemplary disinfection unit 100 having a spherical interior configuration in accordance with one aspect of the present application is depicted in FIG. 5. Similar to the previous FIGURES, the exterior of the disinfection unit 100 may be substantially box-like with a flat bottom so as to be placed on a flat surface; however, the disinfection unit 100 can be any other shape, or combination of shapes, if provided with a means for mounting onto a surface. For example, as shown in FIGS. 1, 2, and 5, the exterior of the disinfection unit 100 is substantially box-like as it gives an overall box-like impression since five out of the six walls essentially form a box shape and the sixth wall, i.e. the top, is partially parallel to the bottom surface except for the slight dome shape appearance. The substantially box-like configuration facilitates placing the disinfection unit against walls or in corners.

The spherical interior configuration further comprises a spherical cavity 108 inside the disinfecting unit 100 for placing item 106 on top of shelf 104. In this embodiment, preferably, the shelf 104 is circular or round to fit the configuration of the spherical cavity 108. In addition, disinfection unit 100 contains computing system 102 allowing the user to view, select, and enter in a variety of options. Unlike the previous FIGURES, however, disinfection unit 100 includes door 202 having a round or curved interior portion. When closed, and as shown below, the door creates a spherical configuration for cavity 108.

Shelf 104 can also be rotated within cavity 108 relative to substantially the entire housing (except the point of rotation) by being mounted on pins 109 at opposite ends of the shelf along a midline m (i.e. diameter) of the shelf 104. In other words, the pins 109 would be positioned a diametrically opposite ends. A motorized mechanism 111 controlled by the computing system 102 may be used to rotate the shelf 104. Other mechanisms can be used to rotate the shelf.

FIG. 6 depicts exemplary placements of illustrative ultraviolet light sources 302 within the spherical configuration of cavity 108 in accordance with one aspect of the present application. Again, item 106 can be placed anywhere on top of shelf 104, the shelf 104 positioned above the bottom of cavity 108. Furthermore, while not shown, a single light source 302 can be used to disinfect or sterilize the entire surface area of item 106.

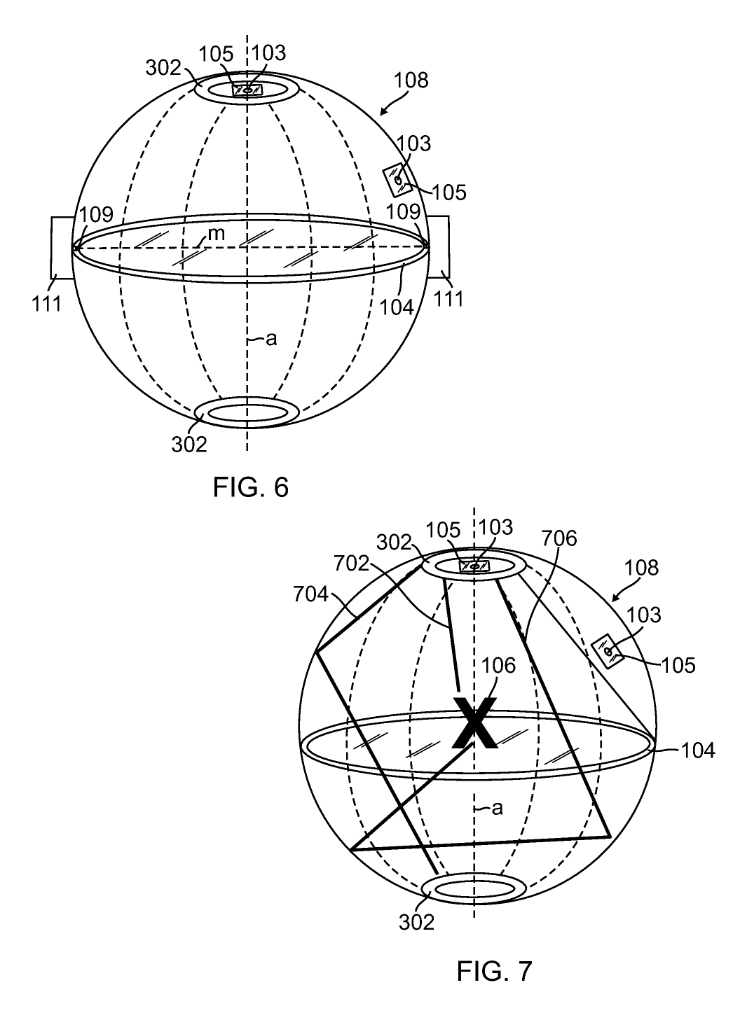

FIG. 7 shows examples of paths taken by ultraviolet light produced from the ultraviolet light source 302 within the spherical configuration in accordance with one aspect of the present application. As shown, a single curved wall defines a spherical cavity 108 having a diameter defining a central axis a. The spherical configuration, as will be shown below, redirects each beam of light to the center making this embodiment more practical for items 106 to be placed on the center of shelf 104. As such, in the preferred embodiment, the central axis a should be unobstructed or devoid of any objects that can either prevent the item 106 from being placed in the center of the sphere or that would obstruct or interfere with the transmission of the ultraviolet light beams from passing through the center of the sphere.

For purposes of illustration, three separate beams 702, 704, and 706 will be followed within the cavity 108 of disinfecting unit 100. Beam 702 begins at ultraviolet light source 302 coupled to the top of cavity 108. Thereafter, beam 702 directly strikes item 106.

Beams may also indirectly strike item 106. Exemplary beam 704 begins at ultraviolet light source 302 coupled to the top of cavity 108. Beam 704 is projected toward the left side of cavity 108. The wall redirects beam 704 towards shelf 104. Because shelf 104 is transparent to the ultraviolet light, beam 704 passes through shelf 104 and strikes the bottom of cavity 108. Beam 704 is redirected again through glass 104 allowing beam 704 to strike the bottom portion of item 106.

In another illustration, beam 706 is projected at shelf 104. Shelf 104 passes beam 706 allowing beam 706 to strike the bottom portion of cavity 108. Thereafter, beam 706 is redirected toward the other bottom portion of cavity 108. Beam 706 is then redirected upwards towards shelf 104. Beam 706, after passing through shelf 104 for the second time, strikes the side of item 106. As a result of the plurality of beams, it can be shown that items 106 placed in the center of cavity 108 are more likely to be struck by the beams in a spherical configuration.

While only a rectangular box configuration and spherical configuration are presented, one skilled in the relevant art will appreciate that cavity 108 of disinfecting unit 100 may include multiple shapes and is not limited to those presented above. For example, cavity 108 may come in a box, columnar, oblong, irregular, or any other shape to disinfect and sterilize items 106.

Furthermore, ultraviolet light sources 302 can be placed throughout the cavity 108 at many different locations. Because of the reflectivity of cavity 108 and the transparency of shelf 104, the ultraviolet light produced by the ultraviolet light sources 302 will eventually strike item 106 within cavity 108.

Ultraviolet light sources 302 can also be interchanged to provide additional types of radiation to item 106. Each source 302 can typically be snapped on and off from cavity 108. As recited above, ultraviolet light sources 302 can produce light having wavelengths from about one hundred nanometers to about four hundred nanometers. More preferably, however, ultraviolet light having wavelengths of about two hundred fifty three point seven nanometers have been used to eliminate microbes. In alternative embodiments, pulsating ultraviolet light sources 302 may be used. Experiments have shown that pulsating ultraviolet light sources 302 more effectively kill bacteria and other microbes that affect items 106. In preferred embodiments, ultraviolet light sources 302 can be made from quartz and a material called soft glass.

In some embodiments, the disinfecting unit 100 further comprises at least one sensor 103. The sensor 103 reads or measures the intensity of the ultraviolet radiation inside the cavity to assure the proper dosage of ultraviolet radiation is administered to item 106. The sensor 103 may be operatively connected to the computing system 102. The computing system 102 can be programmed to alert the user, via an indicator 107, when the intensity of the ultraviolet radiation drops below a specified or predetermined threshold level. The indicator 107 can be in the form of a visual signal, audible signal, or a functional signal (i.e. the ultraviolet lights not turning on), and the like.

In some embodiments, the computing system 102 may adjust the exposure time to the ultraviolet radiation automatically based on the intensity of the ultraviolet radiation detected by the sensor. In other words, if the intensity of the ultraviolet radiation drops below a predetermined threshold, the exposure period may be increased to compensate for the reduced intensity of the ultraviolet radiation. This ensures that the item 106 receives the proper dosage of ultraviolet radiation to disinfect or sterilize the item 106 effectively. The computing system 102 may be pre-programmed to provide a specific period of exposure depending on the intensity of the ultraviolet radiation detected by the sensor 103. For example, the duration of the ultraviolet radiation exposure may be inversely proportional to the intensity of the ultraviolet radiation.

The sensor 103 may be positioned anywhere within the cavity of the disinfection unit 100. Preferably, so as to minimize any obstruction to the redirection of the ultraviolet light transmission, the sensor 103 may be embedded into one of the interior walls of the disinfection unit 100. More preferably, the sensor 103 may be positioned adjacent to the ultraviolet light source 302 in a manner that minimizes interference with ultraviolet light beam's path towards the item 106. For example, in FIG. 3, the sensor 103 may be positioned directly above the upper ultraviolet lights source 302 on the top wall t, or directly below the lower ultraviolet light source 302 on the bottom wall b. Similar positioning can be implemented in the spherical embodiments of FIGS. 6 and 7. Light beams projecting directly upwards or downward, orthogonal to the top or bottom walls t or b, respectively, are unlikely to be redirected in such a way as to hit item 106. Therefore, this type of positioning will least likely hinder the disinfection of item 106.

The sensor 103 may be directly exposed to the ultraviolet light. In some embodiments, the sensor 103 may be protected by a transparent or translucent barrier 105. Like the shelf 104 the transparent or translucent barrier 105 may be made of any material that allows ultraviolet light to pass through.

Disinfecting unit 100 can either come by itself or include other elements which may sterilize or provide other features for a user. For example, disinfecting unit 100 can incorporate a microwave in the cavity 108 of disinfecting unit 100. The microwave would include a dielectric element for heating item 106. Through the dielectric element, microbes on item 106 would be killed. Important, however, is the fact that the reflective walls or coating cannot be made of any type of metal as the microwaves will bounce back to the dielectric element causing damage to disinfecting unit 100. Alternatively, disinfecting unit 100 can incorporate a wash down chamber. The wash down chamber would include a cleansing element for removing dirt or other particles on item 106. One skilled in the relevant art will appreciate that multiple embodiments can be incorporated into disinfecting unit 100.

Furthermore, the basic principles applied in disinfecting unit 100 can be incorporated to other types of devices. One device can include a home countertop disinfection system. This system would have an ultraviolet light source 302 attached to a top portion, the top portion surrounded by reflective walls. The system would thereby be placed over the countertop to disinfect the system. The reflective walls would bounce any ultraviolet light towards the countertop. Alternatively, the system provided above can be applied to any other dirty surface containing microbes or other germs and does not have to be a countertop.

In accordance with one aspect of the present application, a disinfection unit 100 is presented. The disinfection unit 100 includes at least one ultraviolet light source 302 producing ultraviolet light for disinfecting an item 106. In addition, the disinfection unit 100 includes a cavity 108 housing the ultraviolet light source 302, the cavity 108 having a reflective interior for redirecting the ultraviolet light produced by the at least one ultraviolet light source 302. Furthermore, the disinfection unit 100 includes a shelf 104 positioned above a bottom portion of the cavity 108 to support the item 106, the shelf 104 capable of passing light produced by the at least one ultraviolet light source 302 therethrough to disinfect an entire surface area of the item 106.

In accordance with another aspect of the present application, a system 100 for sterilizing an item 106 is presented. The system 100 includes a chamber 108 and a light source located within the chamber 108. In addition, the system 100 also includes a member 104 positioned within the chamber 108 to allow ultraviolet light produced from the light source 302 to disinfect an entire surface area of the item 106 by allowing the ultraviolet light to pass through.

In accordance with yet another aspect of the present application, a method is presented. The method includes placing an item 106 on a surface 104 within a chamber 108 having reflective walls. In addition, the method includes applying ultraviolet light from a single light source 302 to the item 106, wherein the ultraviolet light directly strikes the item 106 and further, indirectly strikes the item 106 through the surface 104 or redirected off the chamber walls allowing the ultraviolet light to eventually strike the item 106 three hundred sixty degrees using the single light source 302.

The foregoing description is provided to enable any person skilled in the relevant art to practice the various embodiments described herein. Various modifications to these embodiments will be readily apparent to those skilled in the relevant art, and generic principles defined herein may be applied to other embodiments. Thus, the claims are not intended to be limited to the embodiments shown and described herein, but are to be accorded the full scope consistent with the language of the claims, wherein reference to an element in the singular is not intended to mean “one and only one” unless specifically stated, but rather “one or more.” All structural and functional equivalents to the elements of the various embodiments described throughout this disclosure that are known or later come to be known to those of ordinary skill in the art are expressly incorporated herein by reference and intended to be encompassed by the claims. Moreover, nothing disclosed herein is intended to be dedicated to the public regardless of whether such disclosure is explicitly recited in the claims.

What is claimed is:

1. A disinfection unit, comprising:
at least one ultraviolet light source for producing ultraviolet light for disinfecting an item;
a housing having a substantially box-like exterior with a flat bottom, the housing defining a cavity to house the ultraviolet light source, the housing having a reflective interior having a spherical configuration with a diameter defining a central axis, wherein the spherical configuration causes the ultraviolet light produced by the at least one ultraviolet light source to be redirected, and wherein the central axis is unobstructed;
a shelf positioned above a bottom portion of the cavity to support the item, the shelf comprising a first flat surface and a second flat surface opposite and parallel to the first flat surface, the shelf capable of passing the ultraviolet light produced by the at least one ultraviolet light source there through to disinfect an entire surface area of the item;
an ultraviolet light sensor positioned on the housing inside the cavity to detect the intensity of the ultraviolet light; and
an indicator operatively coupled to the ultraviolet light sensor to alert when the ultraviolet light is below a predetermined threshold.

2. The disinfection unit of claim 1, wherein the ultraviolet light produced from the at least one ultraviolet light source has a wavelength from about 100 nm to about 400 nm.

3. The disinfection unit of claim 1, wherein the ultraviolet light produced from the at least one ultraviolet light source has a wavelength of about 253.7 nm.

4. The disinfection unit of claim 1, wherein the reflective interior is a thin polished layer of metal.

5. The disinfection unit of claim 1, wherein the reflective interior is a dielectric coating.

6. The disinfection unit of claim 1, wherein the reflective interior is a combination of a thin polished layer of metal and a dielectric coating.

7. The disinfection unit of claim 1, further comprising a door having a reflective interior for sealing the cavity.

8. The disinfection unit of claim 1, wherein the shelf comprises quartz.

9. The disinfection unit of claim 1, wherein the shelf comprises Teflon.

10. A disinfection unit, comprising:
at least one ultraviolet light source for producing ultraviolet light for disinfecting an item;
a housing defining a cavity to house the ultraviolet light source;
a shelf positioned above a bottom portion of the cavity to support the item, the shelf capable of passing the ultraviolet light produced by the at least one ultraviolet light source there through to disinfect an entire surface area of the item; and
an ultraviolet light sensor to detect the intensity of the ultraviolet light.

11. The disinfection unit of claim 10, further comprising an indicator operatively coupled to the ultraviolet light sensor to alert when the ultraviolet light is below a predetermined threshold.

12. The disinfection unit of claim 10, wherein the cavity is spherical in shape.

13. The disinfection unit of claim 10, further comprising a dielectric element for heating the item.

14. The disinfection unit of claim 10, further comprising a cleansing element for removing dirt or other particles on the item.

15. The disinfection unit of claim 10, further comprising a reflective coating to redirect the ultraviolet light produced from the light source to the item.

16. A disinfection unit, comprising:
at least one ultraviolet light source for producing ultraviolet light for disinfecting an item;
a housing defining a cavity to house the ultraviolet light source, the housing having a reflective interior having a spherical configuration with a diameter defining a central axis, wherein the spherical configuration causes the ultraviolet light produced by the at least one ultraviolet light source to be redirected, and wherein the central axis is unobstructed;
a shelf positioned above a bottom portion of the cavity to support the item, the shelf comprising a first flat surface and a second flat surface opposite and parallel to the first flat surface, the shelf capable of passing the ultraviolet light produced by the at least one ultraviolet light source there through to disinfect an entire surface area of the item.

17. The disinfection unit of claim 16, wherein the shelf and the bottom portion of the cavity define a single, sub-cavity of empty space.

18. The disinfection unit of claim 16, wherein the shelf is rotatable relative to substantially the entire housing.

19. The disinfection unit of claim 16, wherein the reflective interior is selected from the group consisting of a thin polished layer of metal and a dielectric coating.

20. The disinfection unit of claim 16, further comprising a door having a reflective interior for sealing the cavity.
